(19) United States
(12) Patent Application Publication
(10) Pub. No.: US 2023/0165558 A1
(43) Pub. Date: Jun. 1, 2023

(54) METHODS AND SYSTEMS FOR HEART SOUND SEGMENTATION

(71) Applicant: Oregon Health & Science University, Portland, OR (US)

(72) Inventors: Clara Mosquera-Lopez, Portland, OR (US); Peter G. Jacobs, Portland, OR (US); Valentina Roquemen-Echeverri, Itagüí (CO); Peter M. Schulman, Portland, OR (US); Stephen Heitner, Portland, OR (US)

(21) Appl. No.: 18/060,933

(22) Filed: Dec. 1, 2022

Related U.S. Application Data
(60) Provisional application No. 63/264,767, filed on Dec. 1, 2021.
(60) Provisional application No. 63/269,094, filed on Mar. 9, 2022.

(51) Int. Cl. A61B 7/04; A61B 5/00

(52) U.S. Cl. CPC A61B 7/04; A61B 5/725; A61B 5/7264

(57) ABSTRACT

Various methods and systems are provided for segmenting heart sounds. In one example, a method includes receiving a phonocardiogram (PCG) signal of a patient, processing the PCG signal to detect a plurality of candidate sounds in the PCG signal, extracting, for each candidate sound, one or more features from the processed PCG signal, entering the one or more extracted features as input to a segmentation model trained to label each candidate sound as an S1 sound, an S2 sound, or neither, receiving output from the segmentation model, and displaying and/or storing the output from the segmentation model.

### CROSS-REFERENCE TO RELATED APPLICATIONS

The present application claims priority to U.S. Provisional Application No. 63/264,767, entitled “AI-POWERED TOOL FOR AUTOMATIC HEART SOUND QUALITY ASSESSMENT AND SEGMENTATION”, and filed on Dec. 1, 2021. This present application also claims priority to U.S. Provisional Application No. 63/269,094, entitled “METHODS AND SYSTEMS FOR HEART SOUND SEGMENTATION”, and filed on Mar. 9, 2022. The entire contents of the above-listed applications are hereby incorporated by reference for all purposes.

### FIELD

The present description relates generally to automatically detecting and categorizing heart sounds.

### BACKGROUND

Cardiovascular disease (CVD) has been the leading cause of death worldwide over the last two decades. Phonocardiogram (PCG), a non-invasive diagnostic method used to record even sub-audible heart sounds, is an effective tool for detecting abnormal heart sounds or murmurs. Obtaining a PCG is less expensive and much faster than obtaining and interpreting an echocardiogram (i.e., heart ultrasound), and could be used to refer patients for additional testing or to a heart specialist if abnormalities are detected.

### BRIEF DESCRIPTION

In one example, a method includes receiving a phonocardiogram (PCG) signal of a patient, processing the PCG signal to detect a plurality of candidate sounds in the PCG signal, extracting, for each candidate sound, one or more features from the processed PCG signal, entering the one or more extracted features as input to a segmentation model trained to label each candidate sound as an S1 sound, an S2 sound, or neither, receiving output from the segmentation model, and displaying and/or storing the output from the segmentation model.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The patent or application file includes at least one drawing executed in color. Copies of this patent or patent application publication with color drawing(s) will be provided by the Office upon request.

The present disclosure will be better understood from reading the following description of non-limiting embodiments, with reference to the attached drawings, wherein below:

FIG. 1 schematically shows an electronic stethoscope in communication with an external computing device.

FIG. 2 shows an example phonocardiogram (PCG) signal.

FIG. 3A shows a flowchart illustrating a method for training a segmentation to segment heart sounds in a PCG signal.

FIG. 3B shows a flowchart illustrating a method for segmenting heart sounds in a PCG signal with a segmentation system trained according to FIG. 3A.

FIG. 4 shows an example set of PCG signals during a pre-processing stage.

FIG. 5 shows an example set of processed PCG signals.

FIG. 6 shows an example processed PCG signal with detected sounds and a plurality of features extracted from the processed PCG signal.

FIG. 7 shows an example multi-layer perceptron network.

FIG. 8 shows an example segmented PCG signal output by a trained machine learning model.

### DETAILED DESCRIPTION

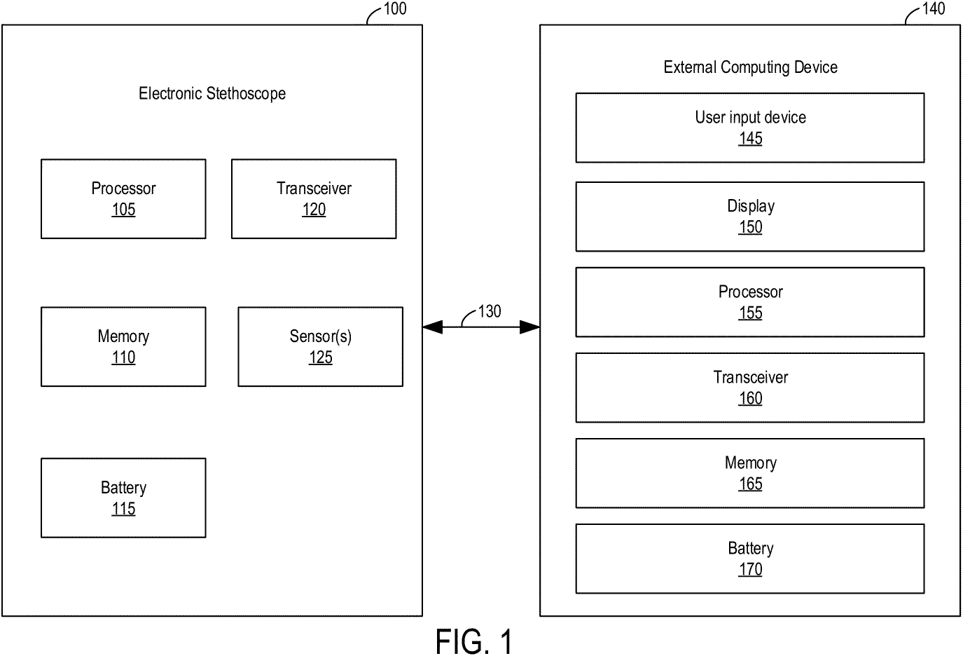

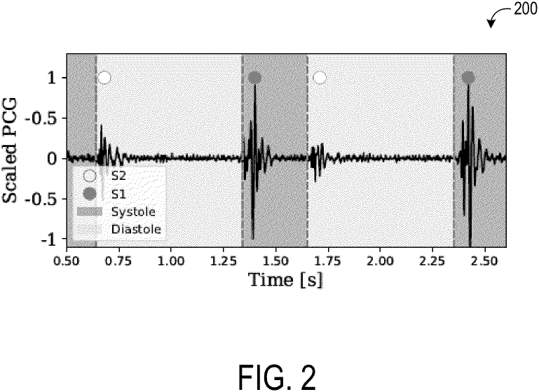

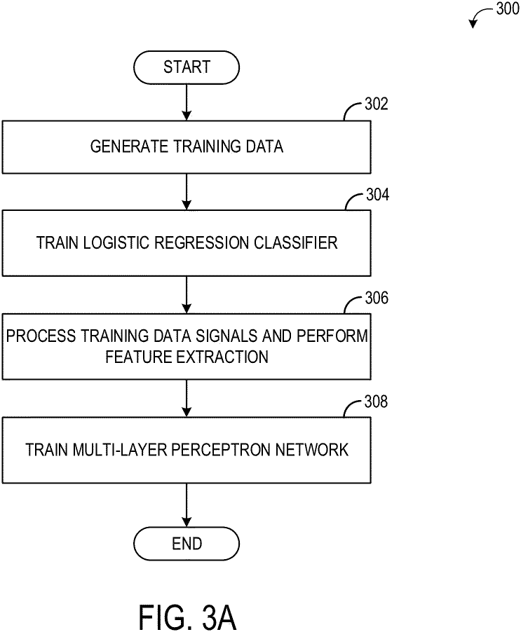

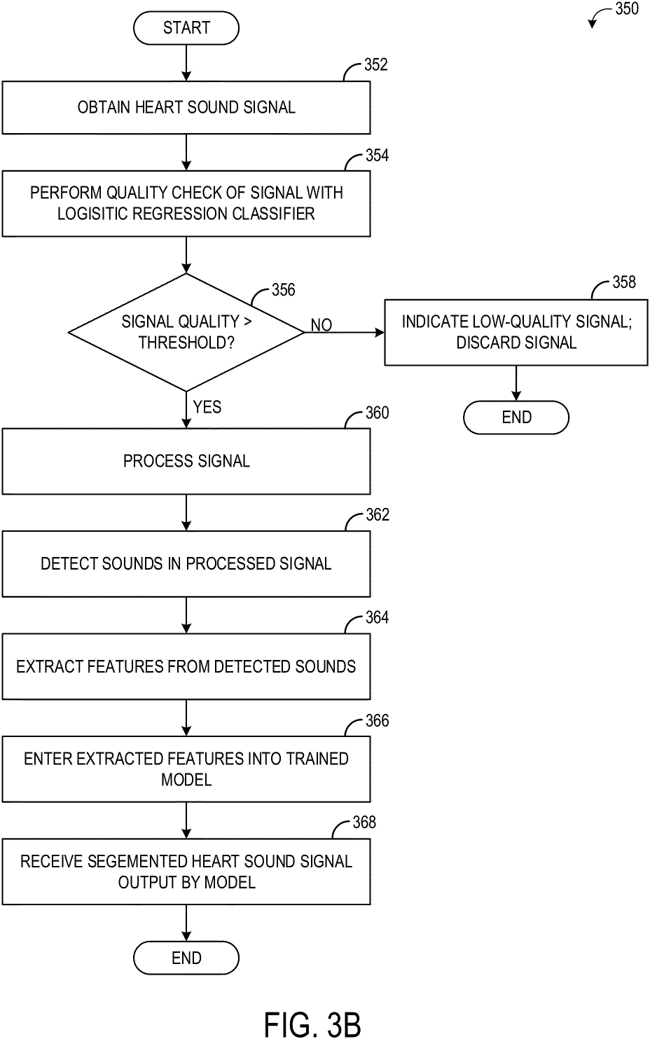

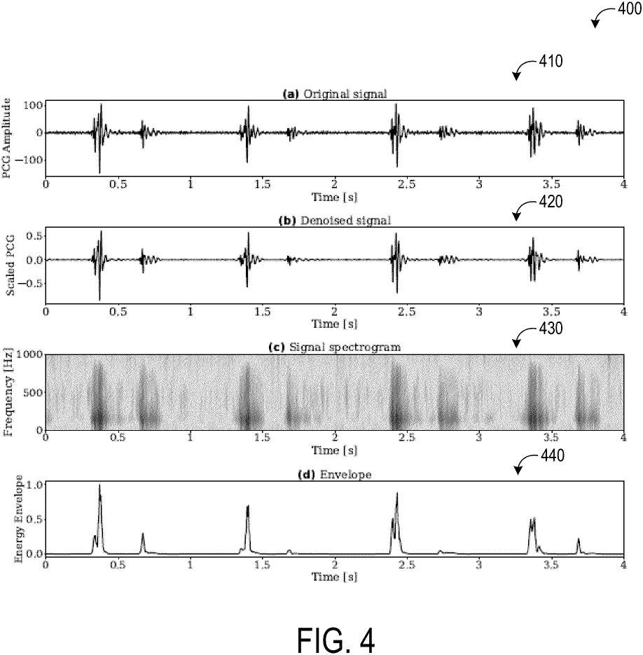

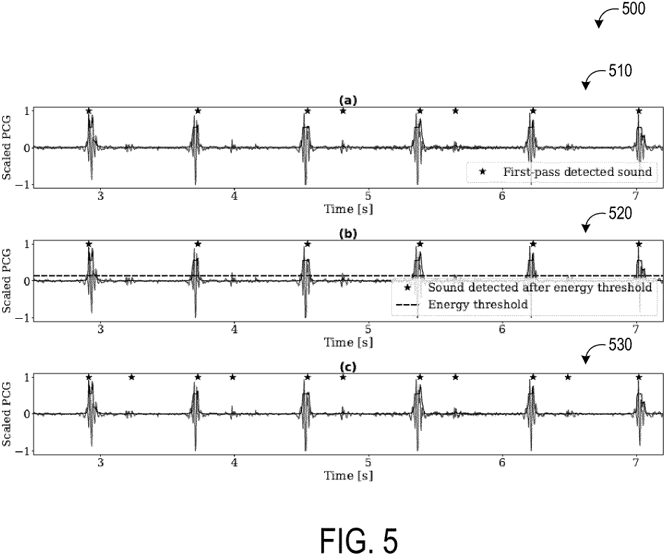

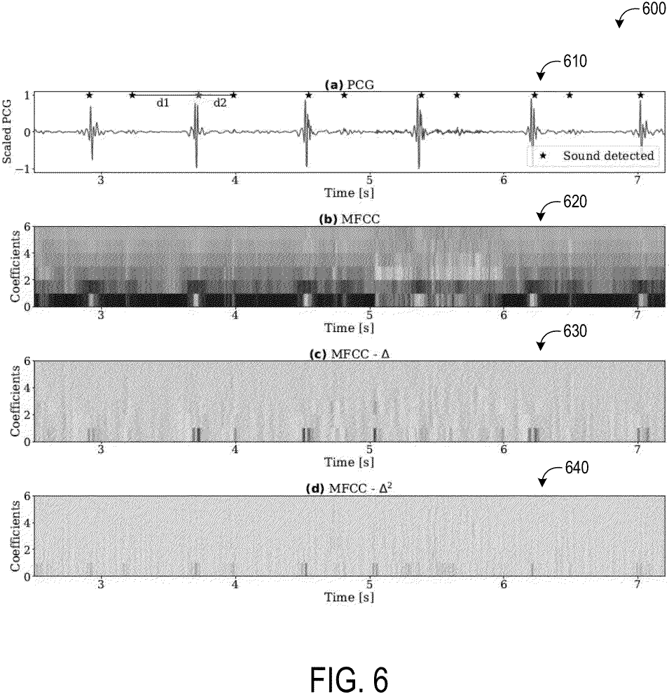

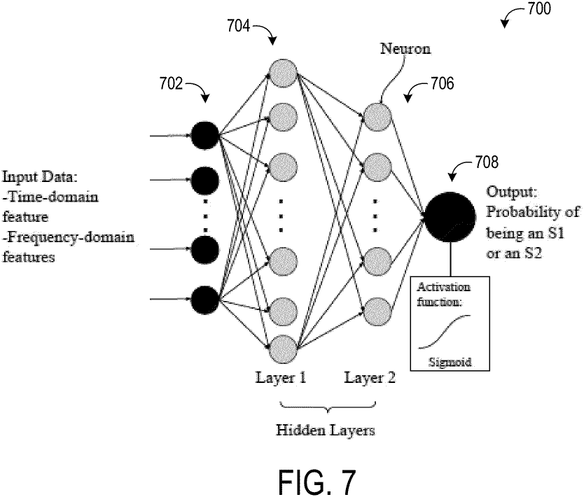

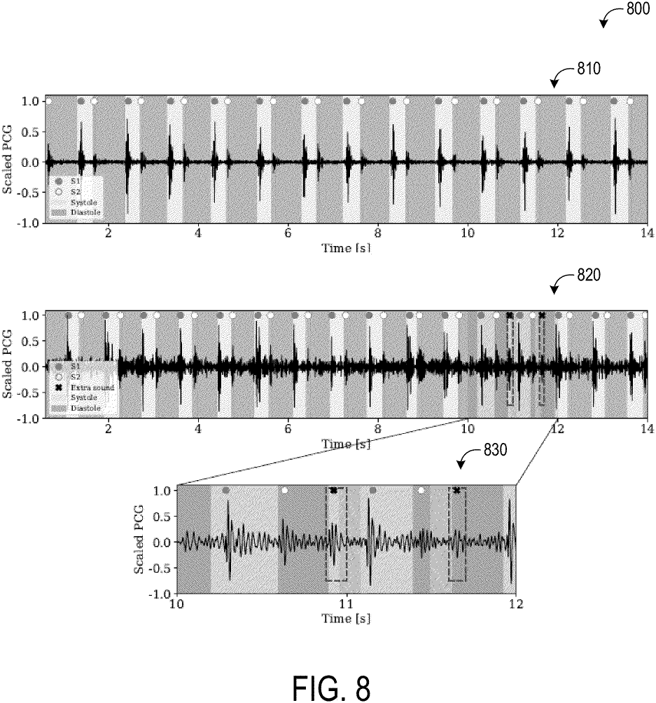

The following description relates to systems and methods for segmenting sounds, such as heart sounds obtained with an electronic stethoscope as shown in FIG. 1. The electronic stethoscope may output the heart sounds in the form of a phonocardiogram (PCG) signal that may be sent to an external computing device, also shown in FIG. 1, for processing in order to segment the heart sounds. The electronic stethoscope may be placed on a subject (e.g., a patient), such as on a skin of the subject, in order to obtain PCG signals, such as the example PCG signal shown in FIG. 2. PCG signals may include two fundamental sounds, S1 and S2, which may be identified automatically using a machine-learning based segmentation system trained according to the method of FIG. 3A and executed during inference according to the method of FIG. 3B. Identifying S1 and S2 in PCG signals may include processing a PCG signal to denoise the signal and generate an energy envelope of the signal, as shown in FIG. 4. After the signal has been processed, sounds are detected in the processed signal, as shown in FIG. 5, and time- and frequency-domain features are extracted from the processed signal, as shown in FIG. 6. The extracted features may be entered as input to a machine learning-based segmentation model, such as the multi-layer perceptron network of FIG. 7. The segmentation model may output an identification of the S1 and S2 sounds in the signal, as shown in FIG. 8.

Turning now to FIG. 1, a schematic of an electronic stethoscope 100 and an external computing device 140 in communication with the electronic stethoscope 100 are shown. For example, the electronic stethoscope 100 may be configured with capabilities of recording various physiological data and communicating with other electronic devices, such as the external computing device 140. The external computing device 140 may be a desktop computer, a laptop computer, a cellular phone, a tablet, or another device that includes a display and a capacity to communicate with other electric devices.

The electronic stethoscope 100 may comprise one or more sensors 125. The one or more sensors 125 may include one or more audio sensors. The one or more audio sensors may each comprise a surface for obtaining audio data, or the one or more audio sensors may include one or more microphones units for collecting audio data. The one or more audio sensors may include or be coupled to an analog-to-digital converter to digitize audio signals detected by the audio sensor, to thereby form an audio transducer. The audio transducer may be used to record physiological sounds from the heart, lungs, stomach, etc. of a patient during an auscultation examination.

In some examples, the one or more sensors 125 may further include other physiological sensors, such as ECG sensors, and/or other suitable sensors such as position sensors.

The electronic stethoscope 100 may comprise a microprocessor or microprocessing unit (MPU) 105, also referred to as processor 105. The processor 105 may be operably connected to a memory 110 which may store machine-readable instructions executable by the processor 105 to control the one or more sensors, store collected data, and/or send the collected data to one or more external devices. Power may be supplied to the various components (the sensors, the microprocessors, the memory, etc.) by a battery 115. The battery 115 may be coupled to charging circuitry, which may be wireless charging circuitry.

The electronic stethoscope 100 may transmit data to the external computing device 140 (e.g., a computing device that is external to the electronic stethoscope 100), another computing device, and/or to a network (e.g., to the cloud). The electronic stethoscope 100 may comprise a transceiver 120, such as a wireless transceiver, to transmit data to the computing device. The transceiver 120 may comprise a Bluetooth transceiver, a Wi-Fi radio, etc. Various wireless communication protocols may be utilized to convey data.

The electronic stethoscope 100 may store data (e.g., audio data) locally on the electronic stethoscope 100. In an example, the data may be stored locally on the memory 110 (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

The electronic stethoscope 100 may be in communication with the external computing device 140 through a communication link 130. The communication link 130 may be a Bluetooth connection, internet connection, radio connection, or another type of connection that allows data to transfer between the electronic stethoscope 100 and the external computing device 140. For example, the electronic stethoscope 100 may record physiological sounds using the audio transducer, and then the transceiver 120 may send the physiological data to the external computing device 140 through the communication link 130. The external computing device 140 may then receive the data by a transceiver 160. The transceiver 160 may comprise a Bluetooth transceiver, a Wi-Fi radio, etc. Various wireless communication protocols may be utilized to convey data.

The external computing device 140 may be a standalone device, as shown. In some embodiments, the external computing device 140 is incorporated into the electronic stethoscope 100. In some embodiments, at least a portion of the external computing device 140 is included in a device (e.g., edge device, server, etc.) communicably coupled to the electronic stethoscope via wired and/or wireless connections. In some embodiments, at least a portion of the external computing device 140 is included in a separate device which can receive PCG recordings from the electronic stethoscope or from a storage device which stores the PCG recordings. The external computing device 140 may include or be operably/communicatively coupled to a user input device 145 and a display 150.

External computing device 140 includes a processor 155 configured to execute machine readable instructions stored in non-transitory memory 165. Processor 155 may be single core or multi-core, and the programs executed thereon may be configured for parallel or distributed processing. In some embodiments, the processor 155 may optionally include individual components that are distributed throughout two or more devices, which may be remotely located and/or configured for coordinated processing. In some embodiments, one or more aspects of the processor 155 may be virtualized and executed by remotely-accessible networked computing devices configured in a cloud computing configuration.

Non-transitory memory 165 may store a segmentation model, a classifier, training data, and/or a training module. The segmentation model may include one or more machine learning models, such as multi-layer perceptron (MLP) networks, comprising a plurality of weights and biases, activation functions, loss functions, and/or gradient descent algorithms, and instructions for implementing the one or more machine learning models to process input PCG signals (e.g., recordings) in order to segment sounds of interest. The segmentation model may include trained and/or untrained models and may further include training routines, or parameters (e.g., weights and biases), associated with one or more machine learning models stored therein. The classifier may include a logistic regression classifier trained to identify if an input PCG signal is a high quality signal or a low quality signal.

The training data may include a plurality of prior PCG recordings/signals and associated ground truth. In some embodiments, the training data may store PCG recordings and ground truth output in an ordered format, such that each PCG recording is associated with one or more corresponding ground truth outputs. The ground truth may include, for each PCG recording, expert-labeled heart sounds (e.g., S1 and S2) and a confidence score for each labeled heart sound (e.g., on a scale of 1-5). The training module may comprise instructions for training one or more of the machine learning models stored as part of the segmentation model and training the classifier. In some embodiments, the training module is not disposed at the external computing device, and thus the segmentation model and classifier each include trained and validated models. In examples where training module is not disposed at the external computing device, the PCG recordings/ground truth output usable for training the segmentation model and classifier may be stored elsewhere.

In some embodiments, the non-transitory memory 165 may include components included in two or more devices, which may be remotely located and/or configured for coordinated processing. In some embodiments, one or more aspects of the non-transitory memory 165 may include remotely-accessible networked storage devices configured in a cloud computing configuration.

User input device 145 may comprise one or more of a touchscreen, a keyboard, a mouse, a trackpad, a motion sensing camera, or other device configured to enable a user to interact with and manipulate data within the external computing device. Display 150 may include one or more display devices utilizing virtually any type of technology. In some embodiments, display 150 may comprise a computer monitor or a smartphone screen. Display 150 may be combined with processor 155, non-transitory memory 165, and/or user input device 145 in a shared enclosure, or may be peripheral display devices and may comprise a monitor, touchscreen, projector, or other display device known in the art.

The external computing device 140 may further include the transceiver 160 for communicating with the electronic stethoscope 100 and/or other devices and a battery 170. It should be understood that the external computing device shown in FIG. 1 is for illustration, not for limitation. Another appropriate external computing device may include more, fewer, or different components.

Thus, the electronic stethoscope 100 may acquire/record heart sounds that may be sent to an external device, such as the external computing device 140, for further processing and/or for display to a user. As explained previously, the heart sounds may be in the form of a PCG signal. An example PCG signal 200 is shown in FIG. 2, with the fundamental sound S1 marked by grey circles and the fundamental sound S2 marked by white circles. The cardiac cycle includes two phases, systole (shown in purple in FIG. 2) and diastole (shown in green in FIG. 2). S1 corresponds to the beginning of systole and results from mitral and tricuspid valve closure. S2 corresponds to the beginning of diastole and results from aortic and pulmonic valve closure. S1 has a lower pitch and is longer than S2. Once S1 and S2 are identified, additional and/or abnormal heart sounds such as S3, S4, murmurs, splits, and gallops can be detected. These abnormalities can be further classified based on when they occur during the cardiac cycle (i.e., during systole or diastole). Abnormal heart sounds can help diagnose and determine the severity of CVD; therefore, accurate detection and classification of abnormal heart sounds is important for diagnosing pathological cardiovascular conditions.

However, accurate detection of the S1 and S2 heart sounds from PCGs of subjects with CVD is challenging due to the presence of extra and/or abnormal heart sounds, such as murmurs, splits, changes in the amplitude or loudness of S1 and S2, and arrhythmias. For instance, S1 could be obscured by mitral regurgitation or aortic stenosis. Also, unlike S1, which is usually heard as a single sound, S2 might be split into its aortic and pulmonic components due respiratory cycle variations.

Moreover, the presence of noise in PCG recordings can complicate accurate localization of S 1 and S2 because the frequencies of some noise sources are similar to those of the fundamental heart sounds. In real-world clinical settings, PCGs can be affected by various types of noise, including sensor and patient motion, physiological sounds, speech, and ambient noise.

Prior approaches for segmenting heart sounds focused implicitly or explicitly on the time-domain regularity of the S 1-S2 and S2-S 1 intervals. For instance, some of these approaches assume that the S1-S2 interval is always shorter than the S2-S1 interval. This assumption might be problematic because diastole sometimes varies with heart rate, while systole remains approximately constant; thus, the duration of diastole and systole might actually sometimes be similar. Also, the assumption that the duration of systole and diastole will remain constant might lead to errors with PCG analysis in a patient with an arrhythmia. Also, measurements of the duration of systole and diastole are prone to high intra- and inter-subject variability.

Thus, according to embodiments disclosed herein, the quality of PCG recordings may be assessed and fundamental S1 and S2 sounds may be identified with a machine learning based system using high quality PCG recordings. The signal quality of the PCG recordings may be determined from a classifier trained using semi-supervised machine learning by mapping PCG time-domain features to the confidence score obtained from expert clinicians to manually label S1 and S2 heart sounds. The fundamental heart sounds S1 and S2 may be detected in high quality PCG signals with a segmentation model, which may be a perceptron network trained to map time and frequency domain features of the PCG signals to expert-labeled S1 and S2 heart sounds. The training data utilized herein (e.g., PCG signals manually labeled by expert clinicians) may include a relatively high level of abnormalities (e.g., 60% of the signals used in the training may include some level of abnormality, relative to other approaches which may rely mostly on normal PCG signals). The proposed segmentation method is robust yet simpler than other state-of-the-art methods such as those employing bidirectional long-short-term-memory (LSTM) neural networks or deep neural network architectures. The proposed segmentation method has an accuracy comparable to more complex published methods using a simple two-layer perceptron classifier with time- and frequency-domain input features that accounts for local contextual sound information.

Turning now to FIG. 3A, an example method 300 for training a machine-learning based segmentation system is shown. At least a portion of the method 300 may be executed on a computing device, such as the external computing device 140 shown in FIG. 1. In some examples, at least a portion of method 300 may be executed on a separate computing device in order to train and validate the classifier and segmentation model as discussed herein, which may then be executed on the external computing device 140. Method 300 may be executed by one or more processors based on instructions stored on memory operatively coupled to the one or more processors.

At 302, training data is generated. The training data may include a plurality of PCG recordings, each recording taken of a different patient. A subset of the PCG recordings (e.g., half or more) may include heart sound abnormalities while the remaining PCG recordings may be considered normal PCG recordings. The training day may include fundamental S1 and S2 PCG heart sounds labeled by experts (e.g., clinicians) with the aid of corresponding ECG reference signals. The experts were also given the option to label extra sounds, such that at least some of the expert labels include heart sounds in addition to S1 and S2 (e.g., S3, murmurs, etc.). For each labeled sound, the experts indicated their confidence on a scale 1- 5 (5 being the highest possible confidence) on the given annotation. Thus, the training data may include PCG recordings and, for each PCG recording, expert-labeled heart sounds (including S 1 and S2) and a confidence level for each labeled heart sound.

At 304, a logistic regression classifier is trained with the training data. The logistic regression classifier, which may be referred to simply as a classifier, may be trained using selected features of the PCG signals in the training data, in order to identify (after training and validation) a signal quality value for each PCG signal.

An auto-correlation function of the spectrogram-based energy envelope of the PCG signals of the training data may be calculated using time frames of five seconds with a three-second overlap. Four features for quality assessment may be computed as the average from all windows. The features were as follows: auto-correlation coefficient (FQ1), estimated cardiac cycle duration (FQ2), standard deviation of the auto-correlation function (FQ3), sum of the absolute value of the auto- correlation function (FQ4). Two additional features were computed from the raw PCG signal: standard deviation of the signal values (FQ5) and root mean square of the first-order signal differences (FQ6). Principal component analysis (PCA) may be employed for dimensionality reduction. Four principal components, which account for more than 97% of variation in the data, may be selected for classification.

To train the classifier, the PCA-transformed features may be mapped to the average confidence of clinicians when annotating the corresponding fundamental heart sounds within a given recording. In some examples, a semi-supervised learning may be used to train the logistic regression classifier using the self-training approach. This method allows the logistic regression classifier to learn from unlabeled data by iteratively predicting pseudo-labels for the unlabeled data. It then adds these classifiers to the training set if the predicted posterior probability of a label given the input features is greater than a pre-defined threshold, which may be empirically set. In one example, the probability threshold is 0.75. PCGs with average confidence level greater or equal than 4 may be considered of high quality and suitable for subsequent analysis, though other confidence levels are within the scope of this disclosure.

At 306, the PCG recordings in the training data are processed to scale and denoise the PCG signals, and feature extraction is performed on the processed PCG recordings. To process the PCG recordings, the raw PCG signals may be band-pass filtered, such as by using a 20-700 Hz sixth level band-pass Butterworth filter for signal conditioning (see FIG. 4). The resulting signal may be linearly scaled to be within the [-1, 1] interval. Wavelet filtering may be applied to the scaled filtered signal to generate denoised signal, for example by using Daubechies db4 wavelet, 5 decomposition levels, and local soft thresholding. An example of a denoised PCG is shown in FIG. 4.

To extract time-frequency descriptors from the denoised PCG signal, a spectrogram S(t, f) of the denoised PCG signal may be calculated using windows of 0.01 s with 50% overlap (see example in FIG. 4). Regions of higher signal energy are red colored, in contrast to blue regions that represent lower energy.

For S1 and S2 detection, the energy envelope Ev(t) of the signal from S(t, f) may be calculated (see example shown in FIG. 4). This envelope was calculated using Equation (3) as the product of the Shannon energy F_{energy}(t) given in Equation (1), along with a frequency-based feature F_{freq}(t) calculated using Equation (2). This enhances the resulting envelope when the frequency with the maximum energy for a time is within the range of 20-200 Hz, which is the frequency range of S 1 and S2.

$${F}_{e n e r g y} \left(t\right) = {\sum }_{f} - S \left(t , f\right) ln \left(S \left(t , f\right)\right)$$

$${F}_{f r e q} \left(t\right) = \frac{{\sum }_{F = 20 H z}^{200 H z} S \left(t , f\right)}{{\sum }_{F = 0 H z}^{700 H z} S \left(t , f\right)}$$

$$E v \left(t\right) = \frac{{F}_{e n e r g y}^{s c a l e d} \left(t\right) \times {F}_{f r e q}^{s c a l e d} \left(t\right)}{m a x \left(\left|{F}_{e n e r g y}^{s c a l e d} \left(t\right) \times {F}_{f r e q}^{s c a l e d} \left(t\right)\right|\right)}$$

In Equation (3), F_{energy}(t) and F_{freq}(t) are scaled by their maximum absolute value to be within the [0,1] interval.

Using Ev(t), peaks that met the following criteria were detected: The amplitude of Ev(t) was greater than AVERAGE Ev(t)) and the distance between consecutive peaks was at least one fourth of the estimated cardiac cycle duration calculated using the PCG autocorrelation function (see FIG. 5).

Extra sounds with low energy may be removed using an energy threshold empirically defined as 10% of the range of energy of detected sounds, plus the value of the energy of the detected sound with the lowest energy.

In some examples, the above-described processing may result in fundamental sounds not being detected or the fundamental sounds being eliminated based on their energy, as in the example shown in FIG. 5. To recover those missing sounds, sounds between detected peaks may be identified that are separated by more than a suitable portion (e.g., 80%) of the estimated cardiac cycle calculated from the envelope auto-correlation function. The portion may be 80% because it is a time lapse in which it may be assumed a fundamental S1/S2 heart sound will be present (see FIG. 5).

The features extracted from the processed PCG signals may include time-domain features and frequency-domain features. In general, as previously mentioned, the diastolic phase of the cardiac cycle has a duration that is greater or equal to that of the systolic phase. Therefore, the distance between detected sounds might be an appropriate feature for differentiating between S1 and S2. However, high inter- and intra-subject variability can diminish the robustness of this feature. For instance, the diastolic phase can vary with heart rate. To make the sound distance more robust, a ratio of distances may be utilized, where the ratio is the ratio of the distance from the sound that is being classified to the next detected sound (d₂) and the distance from the sound that is being classified to the previous detected sound (di), such that F_{dratio} =d₂/d₁ (see example in FIG. 6). Note that, in general, if the sound to be classified is S1, F_{dratio} is approximately less than one, whereas if the sound to be classified is S2, F_{dratio} is greater than one. Thus, the time domain-based features may include the ratio of d₂/d₁.

The frequency domain features may include Mel Frequency Cepstral Coefficients (MFCC). MFCCs are the result of a series of operations: windowed fast Fourier transform (FFT), Mel-filtering, nonlinear transformation, and discrete cosine transform (DCT). The power of this method is that the mel filter transforms the frequencies and generates a representation of sound that is related to human perception of the sound. Heart murmurs as well as S1 and S2 identification is done by physicians by listening carefully to the heart sounds. This is why it makes sense to transform the sounds to MFCCs. MFCCs are a set of static coefficients that are oftentimes used in automated speech recognition algorithms. Dynamic MFCCs of first and second-order can also be calculated, and they are called Δ and Δ², respectively. These dynamic MFCCs represent how the coefficients change from time frame to time frame.

In some examples, 6 static MFCC coefficients, 6 Δ coefficients, and 6 Δ² coefficients (see the examples shown in FIG. 6) may be determined for each time window (e.g., each 0.01 s). A total of 54 MFCC-derived features (static, Δ, and Δ²) may be calculated for the time frame when the sound is being classified (18 features), the time frame of the sound preceding the one being classified (18 features), and the time frame of the sound following the one being classified (18 features).

At 308, a machine learning model, herein a multi-layer perceptron (MLP) network, is trained with the training data (specifically, the extracted features and ground truth). The MLP network is trained to classify S1 and S2 sounds. For the ground truth, S1 was assigned the label 0 and S2 was assigned the label 1. An example high level architecture of the neural network is presented in FIG. 7.

Training the MLP network may include identifying an optimal architecture for the network using cross-validation and by inputting, for each PCG signal of the training data, the extracted features (e.g., the time-domain feature of the distance ratio and the 54 frequency domain features) and associated labels. The optimal architecture may be found by testing various hyper-parameter combinations, such as 576 hyper-parameter combinations, to identify an optimal set of hyper-parameters. An example search space and best parameters found using grid search and optimizing the AUC score are presented in Table 1. Only PCG data with high confidence labels (e.g., heart sounds labeled with confidence greater than 4) were used for the parameter search. The MLP network may be trained using backpropagation, which may adjust connection weights between nodes after each piece of data is processed, based on the amount of error in the output compared to the ground truth.

TABLE 1

| Hyper-parameter | Search space | Best parameter |
|---|---|---|
| Hidden layer sizes: (n1,n2) | n1 = [50,100,150,200] n2 = [-,50,100] | (150,100) |
| Activation function | ReLU and logistic | ReLU |
| Solver | SGD | SGD |
| Batch size | 32 and 64 | 32 |
| Learning rate schedule | Constant and adaptive | Adaptive |
| Initial learning rate | 10⁻³ and 10⁻⁴ | 10-3 |
| Validation fraction | 0.2, 0.25, and 0.3 | 0.2 |

FIG. 3B is a flow chart illustrating a method 350 for deploying a machine learning based segmentation system to automatically identify heart sounds in PCG recordings. The machine leaning based segmentation system may include a classifier and a segmentation model, such as the classifier and segmentation model explained above with respect to FIG. 1, which may be trained according to the method of FIG. 3A. Method 350 may be executed by one or more processors, including a processor of the computing device (e.g., the processor 155 of FIG. 1), based on instructions stored on a memory operatively coupled to each of the one or more processors (e.g., the memory 165 of FIG. 1) and in conjunction with signals received from electronic components of the electronic stethoscope and the computing device.

At 352, a heart sound signal is obtained. The heart sound signal may be a PCG signal of a patient acquired with an electronic stethoscope, for example. At 354, a quality check of the PCG signal is performed with a trained logistic regression classifier. The classifier may assign a signal quality value (e.g., on a scale of 0-100) to the PCG signal based on the learned mapping described above with respect to FIG. 3A. The input into the classifier may include one or more features of the PCG signal selected during training of the classifier. For example, as explained above, six features may be extracted from the PCG signal: the auto-correlation coefficient FQ1, the estimated cardiac cycle duration FQ2, the standard deviation of the auto-correlation function FQ3, the sum of the absolute value of the auto-correlation function FQ4, the standard deviation of the signal values FQ5, and the root mean square of the first-order signal differences FQ6. These six features may be projected into a PCA plane and the four components that account for at least a threshold proportion (e.g., in a range of 90-99%, such as 97%) of the variability are selected. The selected four features (four features selected from FQ1-FQ6 based on the PCA) are entered as inputs into the classifier, which then outputs a quality value.

At 356, method 350 determines if the output signal quality value is greater than a threshold value. The threshold value may be based on the scale of the signal quality values, such as a threshold value of 59 when the scale is 1-100, or the threshold value may be a different value, such as 3 when the scale is different than 1-100 (e.g., when the scale is 1-5, such that all PCG signals having a signal quality value of 60 or higher are considered above the threshold and thus high quality. If the signal quality value is not greater than the threshold, method 350 proceeds to 358 to indicate the PCG signal is a low-quality signal. In some examples, the PCG signal may be discarded and thus not used for further processing. In some examples, a user may be notified (e.g., via a notification displayed on a display of the computing device) that the PCG signal is low quality, and may be prompted to obtain a new PCG signal. Method 350 returns.

If the signal quality value is greater than the threshold, method 350 proceeds to 360 to process the PCG signal. The PCG signal may be processed to generate a scaled, denoised PCG signal, for example by applying a bandpass filter, linearly scaling the signal (after bandpass filtering), and applying a wavelet filter. The denoised signal is then further processed to calculate a spectrogram of the denoised signal and generate an energy envelope of the PCG signal. The process for scaling and denoising the PCG signal as well as calculating the spectrogram of the denoised signal may be the same as the scaling, denoising, and spectrogram calculation process described above with respect to FIG. 3A.

FIG. 4 shows an example set of signals 400 that may be generated during the signal processing described herein. The set of signals 400 includes a first signal 410, which is an example original PCG signal including audio sensor output (in amplitude) over time. The set of signals 400 includes a second signal 420, which is a denoised signal scaled to a range of [-1,1]. The denoised signal may be generated by scaling and wavelet filtering the first signal 410. A third signal 430 of the set of signals 400 is a spectrogram of the denoised signal, calculated using windows of 0.01 s with 50% overlap. The spectrogram is plotted as signal frequency over time, with regions of higher energy in red and regions of lower energy in blue. The set of signals 400 further includes a fourth signal 440, which is an energy envelope of the PCG signal calculated from the spectrogram. The energy envelope (Ev(t)) may be calculated using equations (1)-(3) explained above.

At 362, sounds in the processed signal (e.g., the energy envelope) are detected. The sounds in the processed signal may be detected based on peak amplitudes in the energy envelope. For example, each peak amplitude may be identified, and the peaks having an amplitude greater than the average of the energy envelope and spaced apart from any other peak by at least one-fourth of an estimated cardiac cycle duration may be identified as candidate sounds. Low-energy candidate sounds may be removed by applying an energy threshold, which may be 10% of the range of energy of the detected candidate sounds plus the value of the energy of the detected sound with the lowest energy. Further, any possible missing sounds may be identified by searching for sounds between detected peaks that are separated by more than 80% of the estimated cardiac cycle calculated from the envelope auto-correlation function.

FIG. 5 shows a set of scaled PCG envelope signals 500 during the sound identification process described herein. A first PCG envelope signal 510 includes a first set of detected sounds, indicated by asterisks, that may be identified during a first pass (e.g., peaks having an amplitude greater than the average of the energy envelope and spaced apart from any other peak by at least one-fourth of an estimated cardiac cycle duration). A second PCG envelope signal 520 shows the application of the energy threshold and resultant removal of some of the sounds identified in the first pass. A third PCG envelope signal 530 shows that some of the sounds removed by the application of the energy threshold are added back in due to the sounds being located between peaks that are separated by more than 80% of the estimated cardiac cycle duration.

Returning to FIG. 3B, at 364, method 350 includes extracting features from the detected sounds. The extracted features may include a first, time-domain feature and one or more second, frequency-domain features. The first feature may include a distance ratio (F_{dratio}) for each detected sound, where the distance ratio is a ratio of a distance from the sound that is being classified to the next detected sound (d₂) and a distance from the sound that is being classified to the previous detected sound (d₁), such that F_{dratio} =d₂/d₁. The one or more second features may include frequency-based features for each sound generated via one or more transforms and/or filters. For example, the frequency-based features may include Mel Frequency Cepstral Coefficients (MFCC). To generate the MFCCs, the PCG energy envelope may be transformed to the frequency domain using a windowed fast Fourier transform (e.g., where each window of the signal is transformed to the frequency domain), the transformed signal may be filtered (e.g., using Mel-filtering) to map the powers of the spectrum obtained via the FFT onto the mel scale, a nonlinear transformation may be performed (e.g., taking the logs of the powers at each of the mel frequencies), and a discrete cosine transform may be applied (e.g., to the list of mel log powers). The FFT may be applied on windows of 10 ms with 50% overlap between each window, but other window durations and/or amounts of overlap are within the scope of this disclosure. In addition to the static MFCCs, dynamic MFCCs may also be calculated (e.g., first order and second order coefficients). The MFCCs that are extracted to be used as input for further processing (as explained below) may include, each detected sound to be classified, a plurality of static MFCCs, a plurality of first order coefficients, and a plurality of second order coefficients, such as six of each type of coefficient, for the time frame when the sound to be classified was detected (where the time frame may depend on the duration of the sound with a maximum duration of 100 ms centered at the detected sound peak), as well as a plurality of static MFCCs, a plurality of first order coefficients, and a plurality of second order coefficients for the time frame of the sound preceding the sound being classified and the time frame of the sound following the sound being classified. Thus, in this example, 54 MFCC-derived features may be extracted for each detected sound. The extraction of the MFCCs (static and dynamic) may be performed the same or similarly as the MFCC extraction described above with respect to FIG. 3A.

For example, FIG. 6 shows a set of features 600 extracted from a PCG energy envelope. The set includes a first plot 610 showing a scaled PCG energy envelope with each detected sound marked with an asterisk. For a given detected sound (shown with a red asterisk), a distance ratio (F_{dratio}) is calculated, based on the distance d₁ between the given detected sound and the prior sound and the distance d₂ between the given detected sound and the following sound. A similar distance ratio may be calculated for each detected sound.

The set of features includes a second plot 620 showing a visualization of the static MFCCs calculated for the scaled PCG energy envelope, a third plot 630 showing a visualization of the first order MFC coefficients (Δ), and a fourth plot 640 showing a visualization of the second order MFC coefficients (Δ²).

At 366, the extracted features are entered into a trained segmentation model. The trained segmentation model may be the segmentation model described above with respect to FIG. 1 and/or trained according to method 300. The trained segmentation model may be a multi-layer perceptron (MLP) network. FIG. 7 shows an example architecture of an MLP network 700 according to an embodiment of the present disclosure. The MLP network 700 may include an input layer 702, two hidden layers 704, 706, and an output layer 708. The input layer 702 may be comprised of a plurality of nodes that may take, as input, the first, time-domain feature (e.g., the distance ratio) and each second, frequency-domain feature (e.g., the static, first-order, and second-order MFC coefficients) for each detected sound. The input layer 702 may be connected to the first hidden layer 704, which in turn may be connected to the second hidden layer 706. The hidden layers may be comprised of a plurality of neurons. The second hidden layer 706 may be connected to the output layer 708, which may output a probability that each detected sound is an S1 or an S2 sound. The hidden layers 704, 706 and the output layer 708 may each utilize a nonlinear activation function, such as a sigmoid function or a rectifier linear unit (ReLU). The MLP network 700 may be fully connected, such that each node/neuron in one layer connects with a certain weight to every node/neuron in the following layer.

At 368, method 350 includes receiving the segmented heart sound signal output by the trained segmentation model. The segmentation model, as explained above, takes the time- and frequency-domain features for each detected sound and outputs a probability (e.g., on a scale of 0-1) for each detected sound indicative of whether that detected sound is an S1 or an S2. If the segmentation model outputs a probability that is greater than 0.5 that a detected sound is an S 1, the detected sound may be labeled as S 1. If the segmentation model outputs a probability that is greater than 0.5 that the detected sound is an S2, the detected sound may be labeled as S2. If the segmentation model does not output a probability for either S1 or S2 that is greater than 0.5, the detected sound may not be labeled. The detected sounds may thus be labeled as S1 or S2 (or not labeled) based on the output of the segmentation model. The segmented heart sounds (e.g., S1 and/or S2 labels) may be output for display and/or storage in memory, and may be overlaid on the original PCG signal, at least in some examples. Further, in some examples, the segmented heart sounds may undergo further processing. For example, a post-processing step may be performed to identify possible segmentation errors and extra sounds. For instance, consecutive fundamental sounds of the same type (e.g., two S1s or two S2s in a row) and/or or sounds separated by a distance that is much shorter than the duration of a systole may be flagged or removed.

FIG. 8 shows example segmented heart sounds 800 that may be output by a trained segmentation model, according to method 350 and/or using the MLP network 700. A first plot 810 includes a PCG signal overlaid with S1 and S2 labels according to the output of the segmentation model. For example, the PCG signal may be processed as explained herein to identify detected sounds, features may be extracted for each detected sound and entered into the segmentation model, and the output from the segmentation model may be used to label each detected sound (e.g., as either S1, S2, or no label). The first plot 810 is an example of model output/segmented heart sounds when no additional or abnormal sounds are present. As such, each S 1 and each S2 is correctly labeled by the segmentation model, and no other sounds are included.

A second plot 820 includes a PCG signal overlaid with S1 and S2 labels according to the output of the segmentation model, similar to the first plot 810, but with extra sounds detected by the segmentation model, which are indicated in the red boxes. A third plot 830 is a magnified view of a section of the second plot 820 including the extra sounds. The extra sounds (which are also marked with a black circle) may be classified by the segmentation model as S2 sounds, for example. The post-processing step may determine that the extra sounds are not S2 sounds due to the presence of two S2 sounds in a row and flag the sounds as extra sounds.

Thus, an AI-powered segmentation system for automatic heart sound quality assessment and segmentation is provided herein. The segmentation system includes a classifier trained with semi-supervised learning to map characteristics of a PCG signal to the label confidence given by clinicians when manually annotating PCG recordings. This allows for the evaluation of the quality of PCG recordings and process PCG signals that are of sufficient quality for high confidence segmentation. In some examples, only PCG signals with a threshold quality level are selected for further processing and segmentation. The segmentation system further includes a segmentation model comprising a multi-layer perceptron network trained to output S1 and S2 labels based on time and feature domain characteristics of PCG signals. The segmentation model may be highly accurate, such as an overall cross-validation accuracy of 92% in detecting fundamental S1 and S2 heart sounds. Further details on the cross-validation accuracy, classifier feature selection, segmentation model optimization, and other aspects as described herein are provided in the appendix. Additionally, by including local contextual information of detected sounds surrounding the sound that is being classified in the frequency-domain, the accuracy of PCG segmentation can be improved.

The technical effect of applying the segmentation system including the classifier and segmentation model as disclosed herein to segment fundamental heart sounds is that low quality signals may be identified and discarded such that segmentation may only be performed on high quality signals, which may improve the accuracy of the segmentation. Another technical effect is that a simple, two-layer MLP network may be used to segment the heart sounds, which may utilize a small amount of training data and processing power, allowing the segmentation system to execute on a wide variety of devices in a low-cost manner.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
receiving a phonocardiogram (PCG) signal of a patient;
processing the PCG signal to detect a plurality of candidate sounds in the PCG signal;
extracting, for each candidate sound, one or more features from the processed PCG signal;
entering the one or more extracted features as input to a segmentation model trained to label each candidate sound as an S1 sound, an S2 sound, or neither;
receiving output from the segmentation model; and
displaying and/or storing the output from the segmentation model.

2. The method of claim 1, wherein the one or more features comprise a first, time-domain feature and one or more second, frequency-domain features.

3. The method of claim 2, wherein the first feature comprises, for a selected candidate sound, a distance ratio of a first time duration between the selected candidate sound a previous candidate sound and a second time duration between the selected candidate sound a subsequent candidate sound.

4. The method of claim 3, wherein the one or more second features comprise a plurality of Mel Frequency Cepstral Coefficients (MFCCs).

5. The method of claim 4, wherein the plurality of MFCCs comprise static and dynamic MFCCs.

6. The method of claim 4, wherein the plurality of MFCCs comprise, for a selected candidate sound, a first set of MFCCs calculated over a first time window corresponding to the selected candidate sound, a second set of MFCCs calculated over a second time window corresponding to the previous candidate sound, and a third set of MFCCs calculated over a third time window corresponding to the subsequent candidate sound.

7. The method of claim 1, wherein the segmentation model comprises a multi-layer perceptron network.

8. The method of claim 1, further comprising determining a quality level of the PCG signal with a trained classifier, and wherein processing the PCG signal to detect the plurality of candidate sounds in the PCG signal comprises processing the PCG signal in response to the quality level of the PCG signal being above a threshold quality.

9. The method of claim 8, wherein the classifier is trained to map selected characteristics of the PCG signal to a label confidence given by clinicians when manually annotating PCG recordings.

10. The method of claim 9, wherein the selected characteristics comprise one or more of an auto-correlation coefficient, an estimated cardiac cycle duration, a standard deviation of an auto-correlation function, a sum of an absolute value of the auto-correlation function, a standard deviation of signal values of the PCG signal, and a root mean square of first-order signal differences of the PCG signal.

11. A method, comprising:
receiving a phonocardiogram (PCG) signal of a patient;
determining that a quality level of the PCG signal is greater than a threshold quality level based on output from a trained classifier;
in response to the determination, processing the PCG signal to detect a plurality of candidate sounds in the PCG signal;
extracting, for each candidate sound, a time-domain feature and one or more frequency-domain features from the processed PCG signal;
entering the extracted features as input to a multi-layer perceptron (MLP) network trained to output a label for each candidate sound classifying each candidate sound as an S1 sound, an S2 sound, or neither;
receiving the output from the MLP network; and
displaying and/or storing the output from the MLP network.

12. The method of claim 11, further comprising verifying the output of the MLP network by identifying any consecutively labeled sounds of the same type and/or or labeled sounds separated by a distance that is a threshold amount shorter than a duration of a systole in the patient.

13. A system, comprising:
an electronic stethoscope; and
a processor operatively coupled to a memory storing instructions that, when executed by the processor, cause the processor to:
receive a phonocardiogram (PCG) signal of a patient from the electronic stethoscope;
determine that a quality level of the PCG signal is greater than a threshold quality level based on output from a trained classifier;
in response to the determination, process the PCG signal to detect a plurality of candidate sounds in the PCG signal;
extract, for each candidate sound, a time-domain feature and one or more frequency-domain features from the processed PCG signal;
enter the extracted features as input to a multi-layer perceptron (MLP) network trained to output a label for each candidate sound classifying each candidate sound as an S1 sound, an S2 sound, or neither; and
displaying and/or storing the output from the MLP network.
